(19) United States
(12) Patent Application Publication
(10) Pub. No.: US 2004/0176283 A1
(43) Pub. Date: Sep. 9, 2004

(54) Methods and compositions for the design of synthetic vaccines

(76) Inventors: John A. Robinson, Wermtswil (CH); Gerd Pluschke, Bad Kronzingen (DE); Kerstin Moehle, Wettswil (CH); Bernhard Pfeiffer, Zurich (CH); Rinaldo Zurbriggen, Schmitten (CH); Reinhart Glueck, Bern (CH)

Correspondence Address: PERKINS COIE LLP, POST OFFICE BOX 1208, SEATTLE, WA, 98111-1208, US

(21) Appl. No.: 10/114,918

(22) Filed: Apr. 1, 2002

(51) Int. Cl. A61K 38/16

(52) U.S. Cl. 514/9; 514/12

(57) ABSTRACT

Conformationally constrained peptidomimetics of the Circumsporozoite protein found on the surface of malaria parasites, as well as methods of making the same are provided. These peptidomimetics can be linked to human compatible delivery vehicles for the generation of protective immune responses against malaria.

### FIELD OF THE INVENTION

The invention relates to the fields of structural biology, chemistry and immunology. Specifically, the invention relates to the rational design of synthetic vaccines composed of conformationally constrained peptidomimetics which can be linked to influenza virus-like particles for the efficient generation of pathogen-specific immune responses.

### BACKGROUND OF THE INVENTION

Various publications or patents are referred to in parentheses throughout this application to describe the state of the art to which the invention pertains. Each of these publications or patents is incorporated by reference herein.

Infectious diseases are a major cause of death and debility for many millions of people around the world. One of the world's most devastating infectious diseases is malaria, with more than 2 billion people currently at risk worldwide (Marshall, E.; Science 390:128, 2000), and a toll of several hundred million illnesses, and 1.5-2.7 million deaths annually (WHO, World Health Report, 1998). Despite enormous efforts to control its transmission, malaria is showing signs of resurgence in many parts of the tropics, with continuing transmission in over one hundred countries. Although it has been eradicated from North America, Europe and Russia, the recent importation of malaria to several southern and eastern areas of the United States and Europe indicate the danger malaria continues to pose to nonmalarious countries. Added to the risk of global spread of malaria are the increasing resistance of the parasite to conventional drugs and the insecticide resistance of its carrier, the Anopheles mosquito. Malaria represents a heavy burden on tropical communities, a threat to nonendemic countries, and a danger to travelers.

The malaria parasite has a complex life cycle. When an infected Anopheles mosquito bites a human host, thousands of threadlike sporozoites, the motile microscopic forms of the malarial parasite, enter the blood stream of the host. Within a matter of minutes, the sporozoites are carried to the liver where they rapidly invade liver cells. Without causing symptoms, these sporozoites undergo a radical change and multiply rapidly for the next 4-5 days. Inside the liver cell, a single sporozoite produces between 10,000 and 30,000 daughter merozoites, which cause the infected liver cell to rupture. Tens of thousands of asexual stage merozoites are released from each infected liver cell into the bloodstream, each of which rapidly target and invade a red blood cell. Inside the red blood cell, the merozoites undergo further multiplication. Every few days, the merozoites multiply ten-fold and burst out to infect other red blood cells. This cyclic and massive increase in parasite burden gives rise to the clinical disease recognized as malaria: the parasite eventually colonizes and destroys up to 70% of all red blood cells, causing severe anemia, fever, convulsions, coma and death.

In the absence of immunity or drug treatment, death can occur within hours of noticeable symptoms. If death does not occur and infection continues, some of the parasites further differentiate into a form that is infectious for mosquitoes, thus permitting the life cycle to continue. These parasites, because of their large numbers, can cause particular damage to the nervous system, liver, and kidney. In young children and adults who have not developed natural immunity, this cycle can result in death within hours from cerebral malaria. Others die later in the infection from overwhelming anemia or liver and kidney failure. Four species of malaria infect humans, although only two, falciparum and vivax cause the vast majority of clinical cases and nearly all of the deaths and serious morbidity. Up to 20% of persons infected with falciparum malaria will die if left untreated. Almost all of the serious morbidity caused by falciparum malaria occurs in children under the age of ten, and the impact is especially severe in those under the age of five. Protecting children from malaria is a major goal of current antimalaria research and development.

Despite ongoing development efforts, there is presently no effective drug that reliably prevents malaria. Furthermore, established drugs like chloroquine and proguanil are rapidly losing their effectiveness due to resistance of the parasite, necessitating the administration of newer drugs, such as mefloquine, that may confer some protection against mulidrug-resistant malaria. Most antimalarial drugs currently in use produce side effects that range from unpleasant (nausea, dizziness, fuzzy thinking, disturbed sleep patterns and malaise) to severe clinical and sometimes life-threatening reactions (psychosis, convulsion, encephalopathy). Because of the potential toxicity of chemoprophylactic malaria drugs and the surge of resistant malaria parasites, there is a great need for safer and more reliable prevention or control of malaria infection.

An alternative strategy for the prevention or control of malaria is the development of a vaccine. Vaccines are substances that cause the host's immune system to develop responses that protect against specific diseases. Vaccination represents the most powerful strategy to fight infectious diseases. An effective vaccine stimulates antibody and T cell responses that specifically recognize a particular pathogen and respond quickly to infection, thereby preventing the invader from causing serious clinical disease.

Even after over two decades of effort, there is currently no effective malaria vaccine. Many factors make malaria vaccine development difficult and challenging. To be effective, the vaccine has to target specific points in the complex parasite life cycle during which the organism appears particularly susceptible to the host's immune system. Identification of these targets is the first step in rational vaccine design. While it has been known for some time that sporozoites attenuated by X irradiation can induce a protective immune response against malaria challenge (Miller et al., Science 1986, 234, 1349-1356), isolating and irradiating sporozoites in sufficient quantities for vaccination is an impractical approach to a vaccine for malaria. The production and handling of parasites poses safety hazards and involves the risk of infection due to incomplete attenuation of the live organisms. A safer alternative to sporozoite attenuation would be a vaccine based on a protein subunit of the parasite that is recognized by the immune system, such as a recombinant surface protein or a synthetic peptide.

Several antibody targets on the malaria parasite have been identified, one of which is the circumsporozoite (CS) protein present on the surface of early sporozoites (Potocnjak et al. J. Exp. Med. 1980, 151, 1504-1513). The central portion of the malaria CS protein contains 41 tandem repeats of a tetrapeptide, 37 of which are Asn-Ala-Asn-Pro (NANP) and four of which are Asn-Val-Asp-Pro (NVDP). Many attempts have been made to use this region of the CS protein as a basis for an anti-malaria vaccine. For example, linear, tandemly repeated NANP peptides conjugated to tetanus toxin, have been used in vaccination studies in humans. However, the immune response generated in this way was not strong enough for these conjugates to be useful as a malaria vaccine (Herrington et al., Nature 1987, 328, 257). A number of subsequent approaches were initiated to enhance the immune response to (NANP)ₙ peptides, including the use of a recombinant 40 kDa protein segment, and the incorporation of linear peptides in multiple-antigen peptide (MAP) constructs (Etlinger et al. Eur. J. Immunol. 1991, 21, 1505-1511; Tam et al., J. Exp. Med. 1990, 171, 299-306; Pessi et al. Eur. J. Immunol. 1991, 21, 2273-2276; deOliveria et al., Vaccine 1994, 12, 1012-1017). It is noteworthy that in none of the efforts the conformation of the NANP repeats in the CS protein was taken into account in the design process despite the fact that short linear (NANP)₃ peptides are likely to be unstructured in aqueous solution and susceptible to rapid proteolytic degradation in serum. Furthermore, a later study suggested that a significant part of the immune response against a linear (NANP)₃ peptide is directed against the chain termini (H. M. Etlinger, A. Trzeciak, Phil. Trans Roy. Soc. Lond. B 1993, 340, 69-72), which of course are not present in the native CS protein. It is thus apparent that linear peptides are sub-optimal candidates for vaccination strategies and improved approaches to vaccine design are needed.

One of the major unsolved problems in biology and chemistry is how the amino acid sequence of a protein determines its three-dimensional structure, yet this structure is fundamental to the functioning and mechanism of action of biological processes, such as the recognition by the immune system of invading pathogens. Understanding how proteins fold and the relationship between protein conformation and its recognition by the immune system presents a key challenge in vaccine design. Because peptides can be produced at low cost and at constant quality they are attractive components of synthetic vaccines. However, one of the problems to be overcome is the difficulty of representing in a small peptide the conformational epitopes found on the native antigenic protein that are required for protective antibody responses. Although neutralizing epitopes can often be localized to secondary structures on the surface of a protein, the corresponding regions as linear peptides will be more conformationally mobile and unlikely to adopt to the same extent a stable secondary structure in aqueous solution. Thus, a strategy aimed at reproducing the three-dimensional structures of immunogenic epitopes as they are found in pathogenic proteins in vivo, combined with a suitable delivery system that allows for presentation of the peptidomimetics to the immune system in an undistorted conformation and in multiple copies, would be an important advance in synthetic vaccine design.

### BRIEF DESCRIPTION OF THE DRAWINGS

FIG. 1: This figure shows the chemical structure of a conformationally constrained, template-bound cyclic peptidomimetic having one tetrapeptide motif.

FIG. 2: This figure shows the chemical structure of an optimized conformationally constrained template-bound peptidomimetic having two intact tetrapeptide repeats, indicating the helical turn motif formed by residues 3 through 7.

FIG. 3: This figure shows a three-dimensional model of the distorted helical β-turn region from Asn³ to Asn⁷ in the conformationally constrained, template-bound peptidomimetic containing two intact NPNA units. Color code: blue=nitrogen atoms, red=oxygen atoms, light blue=hydrogen atoms attached to nitrogen atoms, green=chain termini. The Asn⁷ backbone HN group is shown within hydrogen-bonding distance to the Asn³ CO group.

FIG. 4: This figure shows the structural arrangement of a conformationally constrained, template-independent peptidomimetic. The arrow indicates a suitable position for a stabilizing crosslink.

FIG. 5: This figure shows the computer model of a conformationally constrained, template-independent peptidomimetic in a stable conformation displaying tandemly repeated helical β-turns. The arrow indicates a suitable position for a stabilizing crosslink. Color coding: pink=Asn, cyan=Pro, coral=Asn, yellow=Ala. The C(α) atoms are marked with a ball.

FIG. 6: A. This figure shows a representation of superimposed structures taken from a molecular dynamics (MD) simulation in water of residues Asn⁵ through Ala¹⁶ of a conformationally constrained, template-independent peptidomimetic with the repeated helical β-turn supersecondary structure. The “sausage” was calculated using the average displacement of the C(α) atoms, which is represented by the spline radius.

B. A conformationally constrained, template-independent peptidomimetic showing the Apro⁶ (cyan) to Glu¹⁶ (green) crosslink.

C. The conformationally constrained, template-independent peptidomimetic showing average NMR structure deduced in water by NMR and dynamic simulated annealing (SA). The “sausage” now represents the average displacement of the C(α) atoms of four NMR structures. The crosslinked residues are shown in ball-and-stick. The N- and C-terminal NPNA motifs are omitted for clarity. The figure was prepared using MOLMOL (Koradi R. et al., J. Mol. Graph. 1996, 14, 51-55).

Color coding: pink=Asn, cyan=Pro, coral=Asn, yellow=Ala.

FIG. 7: This figure shows the synthesis of an orthogonally protected (2S,3R)-3-aminoproline from the known β-lactam for the internal crosslinking of a conformationally constrained, template-independent peptidomimetic.

i: (Tf)₂O, CH₂Cl₂, pyridine (98%); ii: NaBH₄, THF/DMF (58%); iii: K₂S₂O₈, Na₂HPO₄, MeCN/H₂O (80%); iv: (Boc)₂O, CH₂Cl, DMF, Et₃N (76%); v: LiOH, THF, H₂O (99%); vi: Pd-C, MeOH, H₂ (93%); vii: Fmoc-Osucc, iPr₂NEt, CH₂Cl (68%)

FIG. 8: This figure shows the synthesis of a conformationally constrained, template-independent peptidomimetic.

FIG. 9: This table shows the antibody responses obtained in mice immunized with peptidomimetics coupled to IRIV or adsorbed onto alum. Although all constructs elicit significant ELISA titers, the IRIV-coupled peptidomimetics exhibit superior generation of sporozoite cross-reactive antibodies compared to the alum-adsorbed MAP constructs. Immunogens: 3-alum=conformationally constrained, template-bound peptidomimetic having one tetrapeptide repeat, coupled to a MAP and adsorbed onto alum; 2-IRIV=conformationally constrained, template-bound peptidomimetic having one tetrapeptide repeat, linked to an IRIV; 5-IRIV=conformationally constrained, template-bound peptidomimetic having two intact tetrapeptide repeats, linked to an IRIV.

FIG. 10: This graph shows the anti-peptide mimetic IgG ELISA responses against template-bound peptide mimetic (closed symbols) and template alone (open symbols) in mice immunized three times with the conformationally constrained, template-bound peptidomimetic containing a single tetrapetide repeat conjugated to MAP and adsorbed onto alum (group-a) or the same peptidomimetic linked to an IRIV (group b). Mice were preimmunized with Inflexal™ and received intramuscularly three doses of 50 μg mimetic. Each curve represents the data from an individual mouse. None of the sera exhibited cross-reactivity with the template-MAP construct, which indicates that the immunogenicity of the template itself was negligible.

FIG. 11: This graph shows the mean ELISA (A) and IFA (B) IgG serum responses in mice immunized three times with conformationally constrained, template-bound peptidomimetics. Group a received the template-bound peptidomimetic comprising a single tetrapeptide repeat formulated with alum, groups b and c received the same peptidomimetic linked to IRIV, and group d received the larger loop, conformationally constrained template-bound peptidomimetic comprising two intact tetrapeptide repeats linked to IRIV. Bars represent the mean reactivity plus SE. SE=Standard error.

FIG. 12: This figure shows the distribution of anti-peptide mimetic IgG ELISA and anti-sporozoite IgG IFA titers in ten mice (group d in FIG. 9) immunized three times with the larger loop template-bound peptidomimetic linked to an IRIV after one preimmunization with Inflexal™. ELISA titers correspond to effective dose 20% values calculated using GENESIS LITE software. Every pair of bars represents one animal.

FIG. 13: This table shows the binding properties of peptidomimetic-specific monoclonal antibodies. [a] mAbs were derived from three separate fusion experiments. [b] Mice were immunized with IRIV loaded with the respective mimetics. [c] ELISA reactivity to conjugate 2. [d] ELISA reactivity to conjugate 5. [e] ELISA reactivity to conjugate 8. [f] IFA reactivity to P. falciparum sporozoites. 2=small loop template-bound peptidomimetic-IRIV; 5=larger loop template-bound peptidomimetic-IRIV; 8=template coupled to a MAP.

FIG. 14: This figure shows monoclonal antibody (MAb 1.26, specific for the small loop template-bound peptidomimetic) immunofluorescence labeling of P. falciparum sporozoites using a FITC-labeled secondary antibody. Sporozoites are indicated by white arrows.

FIG. 15: This figure shows the serum IgG titers of BALB/c mice immunized three times with the confonnationally constrained, template-independent peptidomimetic-IRIV. ELISA was performed in ELISA microtiter plates coated either with the conformationally constrained, template-independent peptidomimetic-PE (A), a PE-conjugate of the larger loop template-bound peptidomimetic (B) or with the small loop, template-bound peptidomimetic conjugated to a multiple antigen peptide for coating on the ELISA plates (C) and incubated with serial dilutions of the sera of individual mice. Bound IgG was detected using alkaline phosphatase-conjugated antibodies specific for mouse gamma heavy chains. Note the crossreactivity of anti-template-independent peptidomimetic sera with the larger loop template-bound peptidomimetic (B) and the complete absence of cross-reactivity to the small loop templatebound peptidomimetic (C).

FIG. 16: This figure shows the cross-reactivity of the antibodies generated by the conformationally constrained, template-independent peptidomimetic-IRIV with malaria sporozoites.

Top—Immunofluorescence staining of P. falciparum sporozoites by mouse antiserum against the conformationally constrained template-independent peptidomimetic-IRIV formulation. An FITC-labeled secondary anti-mouse IgG antibody was used.

Bottom—Incubation of the primary antibody with the parasites in the presence of the conformationally constrained, template-independent peptidomimetic (50 μg/ml) abolished staining of the sporozoites.

### DETAILED DESCRIPTION OF THE INVENTION

There is great interest in the use of peptide and protein mimetics in the design of novel synthetic vaccine candidates. Due to their inherent flexibility, linear peptides often elicit antibodies that bind to denatured proteins but that less frequently recognize conformational epitopes in native protein structures. This, together with their often weak ability to elicit antibody production when administered as conjugates in human-compatible adjuvants, for example alum, has so far limited the application of peptides as synthetic vaccine candidates. There is, therefore, increasing interest in the design of constrained synthetic peptide and protein mimetics which accurately reproduce conformational B-cell epitopes present on native pathogenic proteins. Synthetic, conformational B-cell epitopes offer a number of advantages over conventional protein and cellular-based vaccines including the ease of handling and storage of small, inherently stable molecules, potential ease of synthesis, avoidance of problems associated with materials produced in cells, and avoidance of other immune reactions associated with intact foreign proteins.

One approach in the design of peptidomimetics is to attach a short amino acid sequence of interest to a template, to generate a cyclic constrained peptidomimetic. Such cyclic peptidomimetics are not only structurally stabilized by their templates, and thereby offer three-dimensional conformations that may imitate conformational epitopes on viruses and parasites, but they are also more resistant than linear peptides to proteolytic degradation in serum. Another approach to reconstructing biologically relevant conformations through peptidomimetics is to design more complex supersecondary structures, as through the synthesis of longer peptides that can be conformationally constrained by template-independent means. Such longer peptide loops may be more flexible, while still retaining biologically relevant three-dimensional structures. In free solution, such template-independent peptidomimetics may populate the same conformational energy landscapes as epitopes of stable folded proteins.

Thus, the present invention provides a rational structure-based approach to synthetic vaccine design through the use of conformationally constrained peptidomimetics that are structurally optimized to present to the immune system the three-dimensional epitopes found on native antigenic proteins. These peptidomimetics can further be linked to influenza virus like particles, called immunopotentiating reconstituted influenza virosomes (IRIVs) for the efficient generation of immune responses against the native pathogen.

This strategy has several unique advantages over previous approaches: first, the constrained peptidomimetics of the present invention closely resemble the three-dimensional conformations found on the intact pathogenic protein, thus providing improved epitopes for the generation of pathogen-specific antibodies that efficiently cross-react with live pathogens. Secondly, the choice of IRIVs as human compatible immunopotentiating delivery agents capable of presenting the peptidomimetics in multiple copies to the immune system further improves the generation of efficient pathogen cross-reactive antibody responses.

The model system chosen for the practice of the present invention is the conserved central tetrapeptide repeat region of the circumsporozoite (CS) protein of the malaria parasite Plasmodium falciparum. Presently, the three-dimensional structure of the tetrapeptide repeat region in the CS protein is unknown, although theoretical studies suggest it is likely to adopt a stable and repetitious conformation, possibly based on β-helical turns or similar structures. The present invention provides both template-bound and template-independent conformationally constrained peptidomimetics that have been optimized to bear close resemblance to the three-dimensional conformations of the (NANP)ₙ tetrapeptide repeat region of the CS protein of the malaria parasite, as evidenced by the high efficiency of these mimetics to elicit sporozoite cross-reactive antibodies.

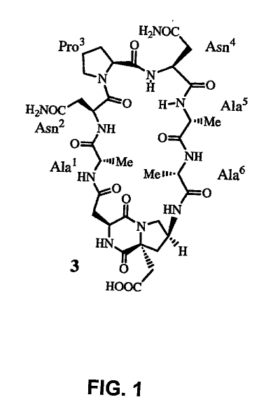

It is still uncertain how multiple tandemly repeated reverse turns based on the NPNA and NVDP cadences might fold into a supersecondary structure in the native CS protein. In this respect, the possibility that the repeat conformational unit is not just the β-turn forming tetrapeptide NPNA, but rather a five-residue NPNAN unit with the Ala residue in the helical region, deserves mention, since this could form the basis for a tandemly repeated conformational unit in the folded CS protein. It has been shown previously that a template-bound cyclic peptide containing the sequence ANPNAA (FIG. 1) elicits some sporozoite cross-reactive antibodies under conditions where a linear peptide containing the same sequence failed to induce a detectable cross-reactive immune response (Bisang, C. et al., J. Am. Chem. Soc., 1998, 120, 7439). While these results established the feasibility of using conformationally constrained peptidomimetics to induce CS protein cross-reactive antibodies, this small loop, relatively rigid peptidomimetic is susceptible of further improvement so as to more efficiently induce cross-reactive antibody responses. First, conformationally constrained peptidomimetics can be optimized structurally in order to elicit cross-reactive antibodies with higher efficiency. Secondly, a suitable antigen delivery system might further improve the titers of cross-reactive antibodies generated by the optimized peptidomimetics.

Accordingly, the present invention provides methods and compositions for the molecular mimicry of the conformational epitopes of the native malaria CS protein by conformationally constrained peptidomimetics. In one preferred embodiment, the invention provides for the emulation of supersecondary structures in the the CS protein based on the NPNA, DPNV tetrapeptide repeats and closely related repeats, including DPNA, and NPNV.

In another preferred embodiment, the invention provides for the emulation of novel helical turn-based supersecondary structures of the NPNA motif by the incorporation of NPNA motifs into peptidomimetics that efficiently elicit sporozoite cross-reactive antibodies.

In one preferred embodiment the invention provides conformationally constrained template-bound cyclic peptidomimetics containing large loops with two or more intact tetrapeptide repeat units. These peptidomimetics very efficiently elicit sporozoite cross-reactive antibodies and may display novel, distorted turn motifs in which one or more amino acid residues may be found in a helical state.

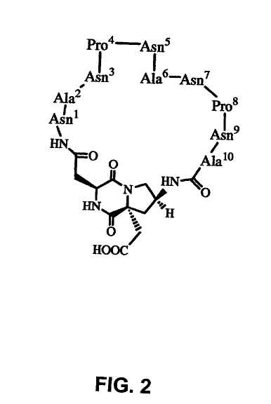

One preferred embodiment of the invention is a conformationally constrained, template-bound peptidomimetic comprising a large loop with two intact NPNA units linked through flanking alanine residues to a template (FIG. 2). This mimetic exhibits in NOESY spectra strong d_{NN}(i,i+1) connectivities between Asn⁵ and Ala⁶ as well as Ala⁶ and Asn⁷, indicative of a helical β-turn within the NPNAN motif. A molecular model of this mimetic, consistent with the NMR data, predicts a helical turn for the NPNAN unit comprising a type-I β-turn, with the Asn³ CO in H-bonding distance of the Ala⁶ HN, and in addition the possibility of an i(Asn³ CO) to i+4 (Asn⁷ HN) hydrogen bond, i.e. with Ala⁶ in the α-region of φ/ψ space. This represents new and valuable information as to how this region might fold in the native CS protein for the design of more effective anti-sporozoite vaccines.

In another preferred embodiment of the invention, molecular modeling is used to construct conformationally constrained peptidomimetics that do not require stabilization by a template. Models of these conformationally constrained peptidomimetics may be assessed for stability and adoption of supersecondary structure in molecular dynamics (MD) simulations in solvent. Adoption of a supersecondary structure by these model peptidomimetics may be evidence that their structures are close to the preferred conformation of the tetrapeptide-repeat region in the native CS protein.

A preferred embodiment of the invention is the construction of larger, more complex conformationally constrained peptidomimetics that exhibit supersecondary structural conformations. Using the backbone φ/ψ angles found in models of other supersecondary structural motifs linear peptides can be built wherein these supersecondary structural motifs with the appropriate backbone φ/ψ angles are tandemly repeated.

In another preferred embodiment the invention provides for the stabilization of a predicted supersecondary structure by appropriate cross-linking of the peptide backbone, and by examining the ability of the resulting cross-linked peptidomimetic to elicit antibodies that recognize the native CS protein on sporozoites. Examination of three-dimensional molecular models can be used to identify suitable cross-links.

In another preferred embodiment the invention provides for the stabilization of the three-dimensional conformation of larger, more complex peptidomimetics. This may be achieved by template-independent means, such as for example the introduction of appropriate crosslinks. Examination and testing of molecular models can be used to determine the appropriate position and type of crosslink that will stabilize the mimetic.

Another preferred embodiment of the invention provides for the synthesis of conformationally constrained cross-linked peptidomimetics by preparation of synthetic amino acids specially adapted for backbone coupling to appropriately positioned amino acids in order to stabilize the supersecondary structure of the mimetics.

In another preferred embodiment, stabilizing crosslinks can be provided by coupling of groups in aminoproline to glutamate or aspartate or other suitable residues. Alternatively, crosslinking can be achieved by coupling free thiol groups of a cysteine to other residues.

In yet another preferred embodiment, the structure of such a complex, conformationally constrained peptidomimetic can be stabilized by a crosslink that involves introducing an amino group at the β-position of a suitably positioned proline and amide coupling to a spatially adjacent side chain carboxyl of glutamate as a replacement for alanine.

In another preferred embodiment of the invention, a substituted aminoproline is provided that contains useful chemical groups in appropriate stereochemical positions for crosslinking.

Another preferred embodiment of the invention provides for the synthesis of the conformationally constrained, template-independent peptidomimetic by preparation of an orthogonally protected (2S,3R)-3-aminoproline from the known β-lactam 6 as shown in FIG. 7. The cross-linked peptidomimetic can be prepared by solid phase synthesis methods, as outlined in FIG. 8. The linear peptide can be assembled using Fmoc-chemistry. Cleavage from the resin and removal of side-chain protecting groups can proceed in one step and the key backbone coupling of the Apro⁶ and Glu¹⁶ side chains can be achieved by cyclization in DMF with HATU.

In another preferred embodiment of the invention, conformational studies are used to determine the preferred solution conformation of the conformationally constrained cross-linked peptidomimetic. The preferred solution conformation can be studied by NMR and MD methods in aqueous solution.

In another preferred embodiment, 2D NOESY spectra of complex conformationally constrained, template-independent peptidomimetics are examined for connectivities between the peptide NH groups in the tandemly repeated helical turns. Such connectivities provide evidence for the relatively stable helical turn formation in the context of a supersecondary structure conformation.

In another preferred embodiment, average solution structures of a complex conformationally constrained, template-independent peptidomimetic are calculated using NOE-derived distance restraints by dynamic simulated annealing and MD simulations. The resulting average structures can reveal expected supersecondary structures deduced from other models.

In another preferred embodiment, the invention provides an approach to synthetic vaccine design which involves using the conformationally constrained peptidomimetics coupled to a human compatible immunopotentiating agent, for the induction of antibody responses against the conformational epitopes of the malaria CS protein. Allied with the use of combinatorial chemistry methods, this approach has great potential for the identification and optimization of molecularly defined synthetic vaccine candidates, in a form directly suitable for human clinical trials.

In a preferred embodiment, the constrained peptidomimetics are coupled to PE or PE′ and then linked to an IRIV. IRIVs are spherical, unilamellar vesicles, prepared from a mixture of phospholipids and influenza virus surface glycoproteins. The hemagglutinin membrane glycoprotein of influenza virus plays a key role in the mode of action of IRIVs. This major antigen of influenza virus is a fusion-inducing component, which faciliates antigen delivery to immunocompetent cells.

In yet another preferred embodiment, antibody responses elicited by IRIVs loaded with the conformationally constrained peptidomimetics are studied in BALB/c mice. Pre-immunization can be achieved with the influenza vaccine Inflexal Bema™ (Bema-Products, Bern, Switzerland). Immunization can be achieved with several doses of IRIV-peptidomimetics.

In another preferred embodiment, the antisera are assessed for the presence of mimetic-specific antibodies, for example by ELISA with conformationally constrained peptidomimetics coated on ELISA plates. The cross-reactivity of these antisera with the other conformationally constrained peptidomimetics can also be analyzed by ELISA. Cross-reactivity of a significant part of the antibody response to an individual conformationally constrained peptidomimetic with another conformationally constrained peptidomimetic can confirm that cross-reacting antibodies recognize supersecondary structures and not peptide chain termini.

Another preferred embodiment of the invention provides for the analysis of the binding of antisera against the conformationally constrained peptidomimetics to the CS-protein. This can be achieved by indirect immunofluorescence assay using P. falciparum sporozoite preparations.

In another preferred embodiment, specificity of cross-reaction is determined by a competition experiment. Abolishment of immunostaining upon incubation of the antiserum with the sporozoites in the presence of a conformationally constrained peptidomimetic is evidence of the generation of a significant proportion of parasite binding antibodies among the total anti-mimetic immune response.

In a preferred embodiment of the invention, the antibody responses elicited by the conformationally constrained cyclic template-bound and template-independent peptidomimetics formulated with alum or with an IRIV are compared.

In another preferred embodiment, the binding properties of mimetic-specific monoclonal antibodies are ascertained. ELISA titers of sera of individual mimetic-IRIV immunized mice that do not correlate strictly with IFA titers indicate that only a subset of antibodies elicited against the peptidomimetics cross-react with the CS protein on the cell surface of the sporozoites. To investigate this further, monoclonal antibodies (mAbs) against the individual mimetics can be generated from hybridoma cell lines and analyzed for their cross-reactivity with malaria sporozoites.

In yet another preferred embodiment, the invention provides an assay to measure the cross-reactivity of antibodies generated by the peptidomimetics of the invention to the CS protein. The cross-reactivity of antibodies generated by the peptidomimetics of the invention can be analyzed by immunofluorescence assays (IFAs) with P. falciparum sporozoite preparations. The IRIV formulation elicits a higher proportion of parasite-binding antibodies among the total antimimetic antibodies than the alum formulation. Peptidomimetic constructs with IFA titers that are significantly higher are superior to others in eliciting a high proportion of parasite crossreactive antibodies.

In another preferred embodiment of the invention, monoclonal antibodies (mAbs) against the individual peptidomimetics are generated. These monoclonal antibodies can be analyzed for their cross-reactivity with malaria sporozoites and have additional useful applications including diagnostics and therapeutics.

The invention thus provides a rational structure-based approach to synthetic vaccine design through the use of a variety of conformationally constrained peptidomimetics that are structurally optimized to present to the immune system the three-dimensional epitopes found on native proteins of the malaria parasite. These peptidomimetics can be linked to immunopotentiating reconstituted influenza virosomes (IRIVs) for the efficient generation of immune responses against the native pathogen.

The conformationally constrained peptidomimetics produced closely resemble the three-dimensional conformations found on the intact pathogenic protein, thus providing improved epitopes for the generation of pathogen-specific antibodies that efficiently cross-react with live pathogens. Furthermore, the choice of IRIVs as human compatible immunopotentiating delivery agents capable of presenting the peptidomimetics in multiple copies to the immune system further improves the generation of efficient pathogen cross-reactive antibody responses.

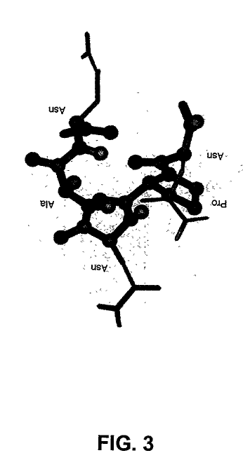

Using molecular modeling methods with geometry optimization, a series of conformationally constrained, template bound molecules incorporating the CS protein tetrapeptide repeat units are constructed. One example of the resulting optimized structures is a cyclic template-bound peptidomimetic containing a large loop with two intact NPNA units (FIG. 2). Interestingly, this mimetic exhibits in NOESY spectra strong d_{NN}(i,i+1) connectivities between Asn⁵ and Ala⁶ as well as Ala⁶ and Asn⁷, indicative of a helical β-turn within the NPNAN motif. A molecular model (FIG. 3) of this novel peptidomimetic, consistent with the NMR data, predicts a helical turn for the NPNAN unit comprising a type-I β-turn, with the Asn³ CO in H-bonding distance of the Ala⁶ HN, and in addition the possibility of an i(Asn³ CO) to i+4 (Asn⁷ HN) hydrogen bond, i.e. with Ala⁶ in the α-region of φ/ψ space.

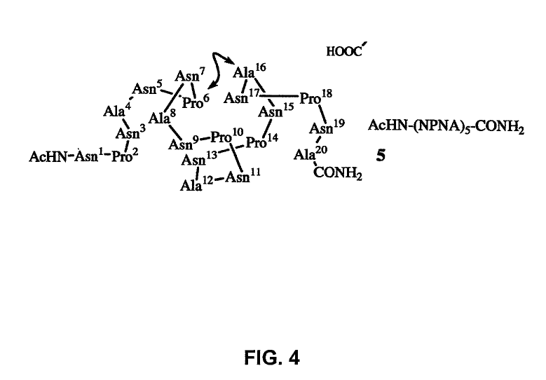

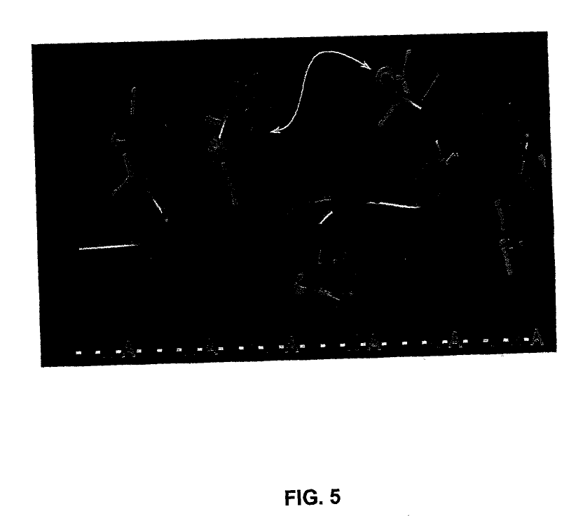

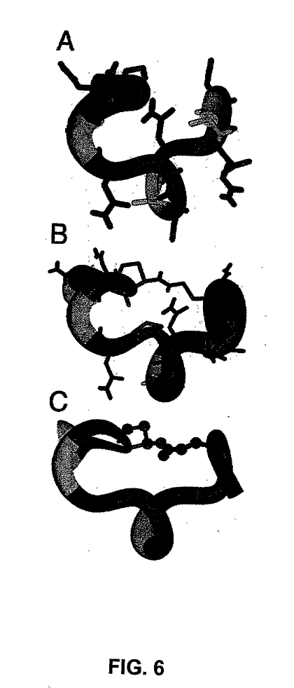

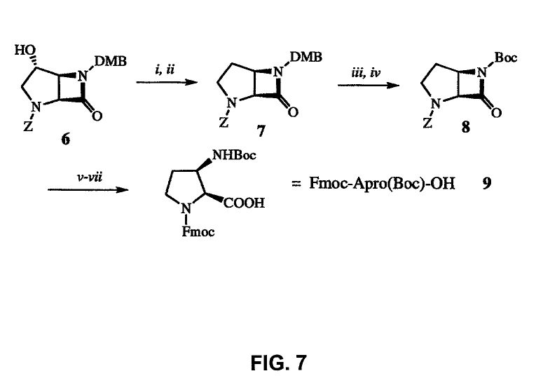

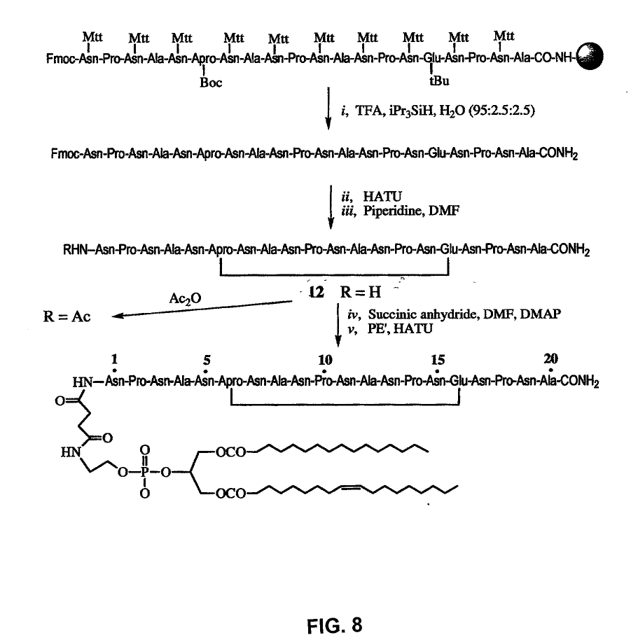

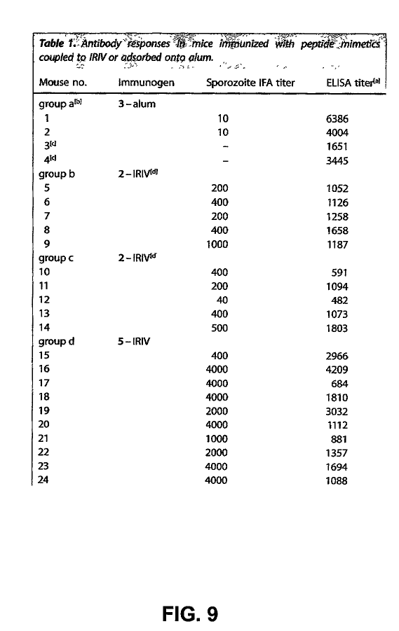

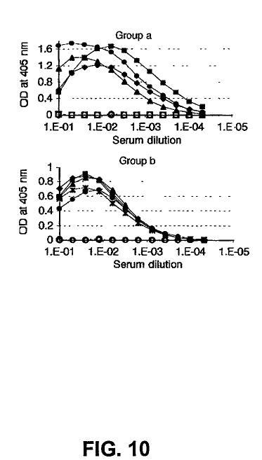

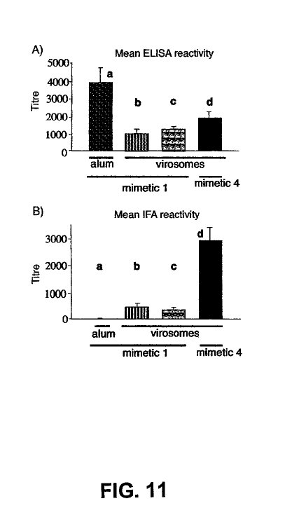

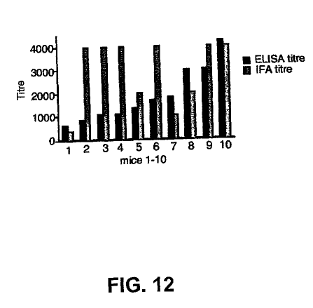

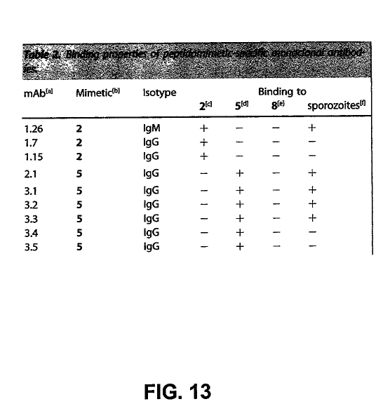

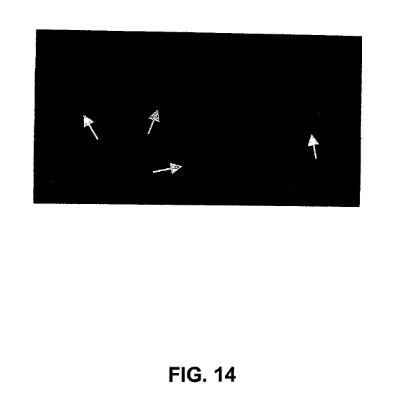

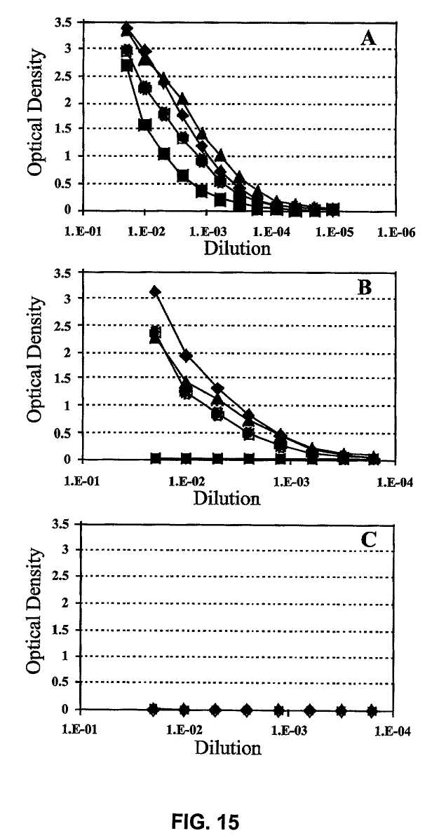

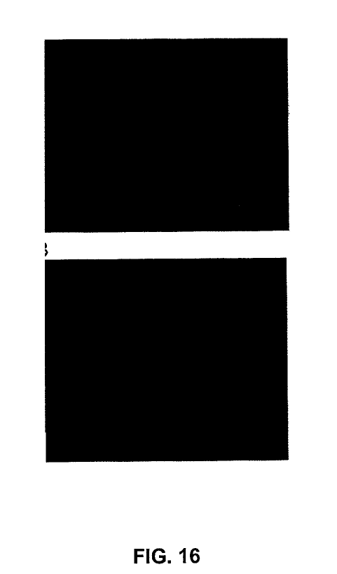

In order to elaborate this observed helical β-turn structure and to further approximate the conformation of the native CS protein with a larger mimetic containing a more complex configuration of motifs, the present invention provides a new approach to peptidomimetic construction. Using the backbone φ/ψ angles for Asn³-Asn⁷ taken from earlier models of the conformationally constrained, template-bound peptidomimetic (FIG. 2), a linear peptide is built with the sequence Ac-(NPNA)₅-NH₂ wherein the helical turn conformation (with the appropriate backbone φ/ψ angles) is also tandemly repeated (FIG. 4). The resulting model of the peptidomimetic (FIG. 5) is stable in MD simulations in water solvent, and adopts the expected repetitious supersecondary structure shown in FIG. 6A. Conceivably, this supersecondary structure may be close to the preferred conformation of the NPNA-repeat region in the native CS protein.

To explore this idea further, the invention provides a strategy to stabilize this supersecondary structure by appropriate cross-linking of the peptide backbone in 5, and by examining the ability of the resulting cross-linked peptidomimetic to elicit antibodies that recognize the native CS protein on sporozoites. Examination of molecular models suggests that one of the suitable cross-links could be formed by introducing an amino group at the β-position of Pro⁶ and amide coupling to the spatially adjacent side chain carboxyl of Glu as a replacement for Ala¹⁶, i.e. as indicated in FIG. 4. A model of this crosslinked peptidomimetic is constructed, and the model also remains in the expected conformation during MD simulations in water (FIG. 6B).

Based on these observations, a conformationally constrained, template-independent peptidomimetic is synthesized. An orthogonally protected (2S,3R)-3-aminoproline is prepared from the known β-lactam (Heinze-Krauss, I. Et al. J. Med. Chem. 1998, 41, 3961-3971) as shown in FIG. 7. The chemistry is straightforward, and the synthesis proceeds in good yields. The cross-linked peptidomimetic is prepared by solid phase synthesis methods, as outlined in FIG. 8. The 20-mer peptide is assembled using Fmoc-chemistry. Cleavage from the resin and removal of side-chain protecting groups proceeds in one step. The key backbone coupling of the aminoproline Apro⁶ (FIG. 7) and Glu¹⁶ side chains is then achieved in a remarkably clean and high yielding cyclization in DMF with HATU. Monitoring the reaction by HPLC shows essentially quantitative cyclization of the precursor. This high efficiency probably reflects the fact that the required conformation is strongly preferred by the peptide backbone.

To verify this prediction, conformational studies of the preferred solution conformation of the conformationally constrained, template-independent peptidomimetic can be undertaken. The peptidomimetic can be studied by NMR and MD methods in aqueous solution at pH 5 and 293K, in analogy to previous studies (Bisang, C. et al., J. Am. Chem. Soc. 1998, 120, 7439-7449). The 1D ¹H NMR spectra indicates the presence of a major conformer and two minor ones (ratio 80:14:6), the latter two most likely arising due to cis-trans isomerism at Asn-Pro peptide bonds, in analogy to earlier studies. The minor forms are not considered further. A full assignment of the ¹H spectrum of the major form is complicated by chemical shift overlap, particularly of the Asn H—C(β) resonances. However, the peptide backbone HN, H—C(α) resonances can be assigned unambiguously.

2D NOESY spectra show strong d_{NN}(i,i+1) connectivities between the peptide NH groups of Asn⁷ and Ala⁸ as well as Ala⁸ and Asn⁹ in the first helical turn, Asn¹¹ and Ala¹² as well as Ala¹² and Asn¹³ in the next helical turn, and between Asn¹⁵ and Glu¹⁶ as well as Glu¹⁶ and Asn¹⁷ in the last helical turn. This, together with the observation of long range NOEs between Pro H C(α) (i+1) and Ala HN (i+3), provide evidence for three relatively stable helical turns formed by the residues Asn⁵-Asn⁹, Asn⁹-Asn¹³, and Asn¹³-Asn¹⁷.

Average solution structures for the conformationally constrained, template-independent peptidomimetic are calculated using NOE-derived distance restraints by dynamic simulated annealing (SA) and molecular dynamics (MD) simulations, using methods described earlier (Bisang, C. et al., J. Am. Chem. Soc. 1998, 120, 7439-7449). The resulting average structures reveal a common core comprising the anticipated three helical turns from Asn⁵-Asn¹⁷, with higher flexibility in the regions of the N-and C-termini (FIG. 6C). The backbone conformation of the central region, however, corresponds closely to the expected supersecondary structure deduced for models of the conformationally constrained, template-independent peptidomimetic (FIGS. 6A and 6B). Therefore, although this novel peptidomimetic is not rigid, it can adopt a supersecondary structure comprising three interlinked helical turns, each based on the (NPNAN) motif.

Thus, the invention provides a series of distinct, structurally optimized conformationally constrained peptidomimetics the immunogenic efficacy of which can be farther enhanced by their combination with an immunopotentiating delivery system. While there are a number of available delivery options, such as liposomes and multiple-antigen peptides (MAPs), immunopotentiating reconstituted influenza virosomes, or IRIVs are particularly well suited for this purpose. IRIVs consist of spherical, unilammelar virus-like particles prepared from a mixture of phospholipids and influenza virus surface glycoproteins, but they do not contain any viral nucleic acids. The hemagglutinin membrane glycoprotein of the influenza virus plays a key role in the mode of action of IRIVs. This major antigen of the influenza virus is a membrane fuision-inducing component, which facilitates antigen delivery to immunocompetent cells. IRIVs are known to act as efficient and highly effective means of enhancing the immune response with an excellent safety profile.

In preparation for immunological studies, the conformationally constrained template-bound peptidomimetic containing a single NPNA motif linked through flanking alanine residues to a template can be coupled through a succinate linker to phosphatidylethanolamine (PE) or a regioisomer (PE′, 1-palmitoyl-3-oleoyl-phosphatidylethanolamine), to afford a conjugate ready for incorporation into IRIVs. In addition, the peptidomimetic can be incorporated into a multiple-antigen peptide (MAP) construct for comparison. Similarly, the conformationally constrained, template-bound peptidomimetic containing a larger loop with two intact NPNA units can also be coupled to PE or PE′ for linkage to an IRIV, and to a MAP. To determine how strongly immunogenic the template is in the conformationally constrained, template-bound peptidomimetics the template by itself can be incorporated into a MAP as a control.

Antibody responses elicited by the MAP constructs adsorbed onto alum, and by IRIVs loaded with the conformationally constrained, template-bound cyclic peptidomimetics containing one or two intact NPNA repeats can be compared in BALB/c mice. After three immunizations, sera from all immunized animals can be collected and assayed for the presence of mimetic-specific antibodies by enzyme-linked immunosorbent assays (ELISAs). Additionally, the cross-reactivity of the sera with the template-MAP construct may be assayed, to verify that the immunogenicity of the template itself is negligible. As shown in FIG. 9, the sera from all immunized mice show significant ELISA titers. None of the sera exhibit cross-reactivity with the template-MAP construct (FIG. 10), indicating that the immunogenicity of the template itself is negligible.

To determine the efficiency of the conformationally constrained, template-bound peptidomimetics in eliciting antibodies that cross-react with malaria sporozoites, immunofluorescence assays can be used. The binding of antibodies to the CS protein is analyzed by immunofluorescence assays (IFAs) with P. falciparum sporozoite preparations. As shown in FIG. 9, the conformationally constrained, template-bound peptidomimetics formulated with IRIVs elicit significant anti-sporozoite responses in all animals immunized. This is in stark contrast to the MAP-alum formulations, where half of the animals immunized with the alum formulation generate no detectable anti-sporozoite antibody response and in the remaining animals the IFA titers are very low. The IRW formulations with the conformationally constrained, template-bound peptidomimetics thus elicit a significantly higher proportion of parasite-binding antibodies among the total anti-peptidomimetic antibodies than the alum formulation.

To ascertain which conformationally constrained template-bound peptidomimetic conjugated to IRIVs more efficiently generates sporozoite cross-reactive antibodies and therefore more closely approximates the native conformation of the malaria CS a comparison between sporozoite IFA titers is informative. As shown in FIG. 11B, the IFA titers of the conformationally constrained, template-bound larger loop (having two intact NPNA repeats) peptidomimetic conjugated to IRIV are significantly higher than those of the more rigid, smaller loop template-bound peptidomimetic, even though their ELISA reactivity differ only slightly. The larger loop, conformationally constrained template-bound peptidomimetic is thus superior to the smaller loop conformationally constrained template-bound peptidomimetic in eliciting a high proportion of parasite cross-reactive antibodies.

Because the ELISA titers of sera of individual peptidomimetic-IRIV immunized do not correlate strictly with IFA titers (FIGS. 9 and 12), it is likely that only a subset of antibodies elicited against the peptidomimetics cross-react with the CS protein on the cell surface of the sporozoites. To investigate this further, monoclonal antibodies (mAbs) against both the smaller and larger loop template-bound peptidomimetics can be generated. Three hybridoma cell lines secreting monoclonal antibodies against the smaller loop template-bound peptidomimetic linked to an IRIV, and six lines secreting monoclonal antibodies against the larger loop template-bound peptidomimetic linked to an IRIV are isolated. The cross-reactivities of the monoclonal antibodies produced by the three clones specific for the smaller loop template-bound peptidomimeti-IRIV (designated mAbs 1.7, 1.15, and 1.26) and by the six clones specific for the larger loop template-bound peptidomimetic-IRIV (designated mAbs 2.1, 3.1, 3.2, 3.3, 3.4 and 3.5) are analyzed as shown in FIG. 13. One of the monoclonal antibodies against the smaller loop template-bound peptidomimetic-IRIV (mAb 1.26) and four of the monoclonal antibodies against the larger loop template-bound peptidomimetic-IRIV (mAbs 2.1, 3.1, 3.2, and 3.3) cross-react with P. falciparum sporozoites in IFAs as shown in FIG. 14. All monoclonal antibodies bind to the peptidomimetic used for immunization but not to the respective second peptidomimetic or to the template conjugated to a MAP (FIG. 13).

To ascertain the relationship between the conformation of the larger loop template-bound peptidomimetic and its increased efficiency in eliciting sporozoite cross-reactive antibodies, the three-dimensional structure of the larger loop template-bound peptidomimetic in solution is determined. The conformation of this peptidomimetic in aqueous solution can be investigated by NMR spectroscopy at 290 and 300 K and pH 5.0). The one-dimensional NMR spectra indicate the presence of one major conformer and two minor ones (in a ratio of 77:15:8) with the latter two most probably arising due to cis-trans isomerism at Asn-Pro peptide bonds, in analogy to earlier studies. The minor forms are not considered further. Although the peptide backbone groups (NH, C(α)H) could be assigned, extensive overlap prevented residue-specific assignments of side-chain Asn resonances. This, together with a sparcity of long-range NOEs, thwarted attempts to calculate solution structures based on NOE restraints.

Nevertheless, NOESY spectra reveal strong d_{NN}(i,i+1) connectivities between Asn⁵ and Ala⁶, as well as between Ala⁶ and Asn⁷. These, together with high field shifted resonances and a relatively low temperature coefficient for the Ala⁶ NH group (δ=7.82 and δ/Δ=3.7 ppb K-1) suggest that a β-turn is formed by the four residues Asn³-Pro⁴-Asn⁵-Ala⁶. A β-turn structure, however, may not be the whole story. The d_{NN}(i,i+1) connectivities show that the Ala⁶ NH group is close to the peptide NH group of the preceeding Asn (as expected in a β-turn) and the following Asn residue. This could occur if the Ala⁶ residue is in the α region of φ/ψ space, with the Asn³ CO moiety within (or close to) hydrogen-bonding distance of both the Ala⁶ NH and the Asn⁷ NH groups as shown in the model in FIG. 3. This leads to the intriguing possibility that conformations are present in which a (perhaps distorted) β-turn is extended by one residue to create a five-residue conformational unit (NPNAN) with Ala in a helical state. This type of helical turn has not been observed in solution conformation studies of the small loop template-bound peptidomimetic. This novel supersecondary structure may explain the increased efficiency of the larger loop template-bound peptidomimetic in eliciting sporozoite cross-reactive antibodies.

To further investigate the impact of this novel supersecondary structure on the efficiency of peptidomimetics in eliciting sporozoite cross-reactive antibodies, the previously constructed conformationally constrained, template-independent peptidomimetic comprising an internal crosslink for stabilization of the three interlinked helical turns (FIGS. 5 and 6) can be coupled to PE or PE′ as shown in FIG. 8, and then linked to an IRIV. Antibody responses elicited by IRIV loaded with this conformationally constrained, template-independent peptidomimetic-PE conjugate (14 in FIG. 8) can be studied in BALB/c mice. After a preimmunization with the influenza vaccine Inflexal Berna™ (Berna-Products, Bern, Switzerland), and three doses of the IRIV-conformationally constrained, template-independent construct, the sera of all immunized mice contain mimetic-specific antibodies, as demonstrated by ELISA with the conformationally constrained, template-independent peptidomimetic coated on ELISA plates (FIG. 15A).

The cross-reactivity of these anti-sera with the template-bound small-and larger loop peptidomimetics can also be analyzed by ELISA. The sera from three of four mice immunized with the conformationally constrained, template-independent peptidomimetic-IRIV cross-react with the larger loop peptidomimetic (FIG. 15B), but none react with the small loop, relatively rigid template-bound peptidomimetic (FIG. 4C). It is interesting to note that a helical NPNAN turn is possible in the template-bound larger loop mimetic but not in the template-bound, relatively rigid small loop peptidomimetic. That a significant part of the antibody response to the more complex, conformationally constrained, template-independent peptidomimetic-IRIV cross-reacts with the larger loop template-bound cyclic peptidomimetic also means that these cross-reacting antibodies do not recognize alone the ends of the peptide chain in the template-independent peptidomimetic, but rather the novel helical-turn supersecondary structure in the central part of the molecule.

To determine the sporozoite-crossreactivity of the antibodies elicited by the conformationally constrained, template-independent peptidomimetic, the binding of anti-conformationally constrained, template-independent-IRIV antisera to the CS-protein can be analyzed by an indirect immunofluorescence assay using P. falciparum sporozoite preparations. As shown in FIG. 16A, a significant anti-sporozoite antibody response is detected in all immunized animals. To further examine the specificity of the cross-reaction a competition experiment can be performed. Incubation of the antiserum with the sporozoites in the presence of the conformationally constrained, template-independent peptidomimetic completely abolishes immunostaining (FIG. 16B). The IRIV formulations with this complex, conformationally constrained, template-independent peptidomimetic thus elicit a significant proportion of parasite binding antibodies among the total anti-mimetic immune response.

The IRIV delivery system of the invention more efficiently elicits parasite cross-reactive antibodies compared to alum-adjuvanted constructs of the peptidomimetics with a multiple-antigen peptide (MAP). The approach is well suited for the design of molecularly defined synthetic vaccine candidates, in a form that is directly suitable for human clinical testing. Like liposomes, virosomes can be used to deliver therapeutic substances to a wide variety of cells and tissues, but unlike liposomes, virosomes offer the advantage of efficient entry into the cells followed by the intracellular release of the virosomal contents triggered by the viral fusion protein. Moreover, due the incorporation of active viral fusion proteins into their membranes, virosomes release their contents into the cytoplasm immediately after being taken up by the cell, thereby preventing the degradation of the therapeutic substance in the acidic environment of the endosome (U.S. Pat. No. 6,040,167).

The presentation of the peptidomimetics on the surface of an IRIV, in an undistorted conformation, and in multiple copies, is ideally suited to allow cross-linking of surface Ig receptors and generate an efficient antiparasite-directed immune response. The methods of the invention demonstrate that immunization with the small loop, template-bound peptidomimetic-IRIV formulation induces anti-sporozoite responses that are superior to those elicited by an alum-adjuvanted peptidomimetic-MAP construct. The alum-adjuvanted small loop template-bound peptidomimetic formulation induces high titers of antimimetic antibodies but hardly any sporozoite cross-reactive immune response. Alum, the adjuvant most commonly used for vaccines in humans, thus apparently favors the generation of antibodies against conformations of the NPNA motif that do not resemble the native CS protein.

The larger loop, template-bound peptidomimetic-IRIV immunogen elicits significantly higher sporozoite cross-reactive IFA titers than the small loop template-bound peptidomimetic-IRIV immunogen. However, antimimetic ELISA titers are comparable with both constructs. This demonstrates that the larger loop template-bound peptidomimetic elicits a higher proportion of sporozoite cross-reactive antibodies in the total antimimetic antibody population than does the small loop template-bound peptidomimetic. This conclusion is strengthened by the properties of the antimimetic monoclonal antibodies isolated here, which demonstrate that only a portion of the antimimetic antibodies are sporozoite cross-reactive and confirm that the larger loop template-bound peptidomimetic has superior immunogenic properties. Although the small and large loop peptidomimetics analyzed are closely related in sequence, none of the antimimetic mAbs generated cross-react with both structures. This indicates that the relevant conformational epitopes presented by the two structures are significantly different from each other, but close enough to the conformation(s) of the CS-protein repeat unit to elicit sporozoite cross-reactive antibodies. Interestingly, NMR studies of the mimetic 4 suggest that helical-turn conformations (FIG. 3), based on the five-residue NPNAN motif, may be present in the large loop template-bound peptidomimetic but not in 1. Finally, the results also demonstrate that the template structure used has negligible immunogenicity.

The IRIVs can in principle be loaded simultaneously with several different peptidomimetic B-cell epitopes and with linear peptides as T-cell epitopes (Poltl-Frank, F. et al., Clin. Exp. Immunol., 1999, 117, 496; Moreno, A. P. et al., J. Immunol., 1993, 151: 489) including universal T-helper cell epitopes (Kumar, A. et al., J. Immunol. 1992, 148, 1499-1505) and others known to those of skill in the art. Furthermore, separate IRIV-peptidomimetic and IRIV-T-cell epitope constructs may be combined to produce multi-component vaccines.

Based on these results, IRIVs appear to have great potential in the design of molecularly defined combined synthetic vaccines, including those targeted against multiple antigents and development stages of P. falciparum, or indeed against other infectious agents. Furthermore, an IRIV-based synthetic peptide vaccine would be expected to be safe, since IRIV-based protein vaccines have already shown a very good safety profile in humans (Glueck, R., Vaccine 1999, 17, 1782). The concerted application of combinatorial peptidomimetic chemistry with the use of IRIVs as an efficient human-compatible delivery system, may prove to be of great value in the design of molecularly defined synthetic peptide vaccines against a wide variety of infectious diseases.

Conclusion

Synthetic linear peptides are often compromised as vaccine candidates due to their inherent flexibility and susceptibility to proteolysis. Linear peptides often elicit antibodies that bind well to denatured proteins, but less frequently recognize conformational epitopes in native protein structures. A further problem is the weak immune responses elicited by linear peptides, even conjugated to carrier proteins, when administered in alum, the commonly used human compatible adjuvant.

The present invention provides an approach to synthetic vaccine design in which cyclic peptidomimetics are presented to the immune system in multiple copies on the surface of Immunopotentiating Reconstituted Influenza Virosomes (IRIVs). These virosome particles contain also influenza virus proteins that facilitate uptake of the virosome by immunocompetent cells. IRIVs have been licensed already for human use, so the peptidomimetics can be tested in a format that directly allows human clinical studies.

The invention provides peptidomimetics of the central (NPNA)ₙ repeat region of the circumsporozoite (CS) protein of the malaria parasite Plasmodium falciparum, as well as of closely related repeats, including DPNV, DPNA, and NPNV. Previous NMR and modeling studies suggest that NPNAN units in this region adopt a helical β-turn, which may be tandemly repeated to form a novel supersecondary structure. To test this proposal, cyclic template-bound and larger, more complex template-independent peptidomimetics are prepared, and shown by NMR methods to adopt preferred conformations having three tandemly repeated helical turns. Antibodies raised against the mimetics cross-react with the native CS protein on P. falciparum sporozoites.

In general, the methods and compositions of the present invention offer great potential for the design of molecularly defined combined synthetic vaccines, including those targeted against multiple antigens and development stages of P. falciparum, and against other infectious agents.

Definitions

Amino acids and amino acid residues described herein may be referred to according to the accepted one or three letter code referenced in text books well known to those of skill in the art, such as Stryer, Biochemistry, 4^{th} Ed., Freeman and Co., New York, 1995 and Creighton, Proteins, 2^{nd} Ed. Freeman and Co. New York, 1993.

As used herein, the terms “peptide” and “polypeptide” are used synonymously and in their broadest sense to refer to a compound of two or more amino acid residues, or amino acid analogs. The amino acid residues may be linked by peptide bonds, or alternatively by other bonds, e.g. ester, ether etc. As used herein, the term “amino acid” or “amino acid residue” refers to either natural and/or unnatural or synthetic amino acids, including both the D or L enantiomeric forms, and amino acid analogs.

The term “epitope” or “B cell epitope” as used herein, designates the structural component of a molecule that is responsible for specific interactions with corresponding antibody (immunoglobulin) molecules elicited by the same or related antigen. More generally, the term refers to a peptide having the same or similar immunoreactive properties, such as specific antibody binding affinity, as the antigenic protein or peptide used to generate the antibody. An epitope that is formed by a specific peptide sequence generally refers to any peptide which is reactive with antibodies against the specific sequence.

The term “antigen” as used herein, means a molecule which is used to induce production of antibodies. The term is alternatively used to denote a molecule which is reactive with a specific antibody.

The term “immunogen” as used herein, describes an entity that induces antibody production in a host animal. In some instances the antigen and the immunogen are the same entity, while in other instances the two entities are different.

The term “immunopotentiating” is used herein to refer to an enhancing effect on immune functions which may occur through stimulation of immune effector cells and may lead to increased resistance to infectious or parasitic agents.

The term “subunit vaccine” is used herein, as in the art, to refer to a vaccine that does not contain whole parasites, but rather contains one or more proteins derived from the parasite or fragments of parasitic proteins.

The term “peptidomimetic” is used herein to denote a peptide or peptide analog that biologically mimics active determinants on parasites, viruses, or other bio-molecules.

The term “conformation” as used herein denotes the various nonsuperimposable three-dimensional arrangements of atoms that are interconvertible without breaking covalent bonds.

The following examples are provided to better illustrate the claimed invention and are not to be interpreted as limiting the scope of the invention. To the extent that specific materials are mentioned, it is merely for purposes of illustration and is not intended to limit the invention. Unless otherwise specified, general chemical and peptide synthesis procedures, such as those set forth in Voet, Biochemistry, Wiley, 1990; Stryer XXX; Peptide Chemistry. A Practical Textbook, 2nd ed., Miklos Bodanszky, Springer-Verlag, Berlin, 1993; Principles of Peptide Synthesis, 2nd ed., Miklos Bodanszky, Springer-Verlag, Berlin, 1993; Chemical Approaches to the Synthesis of Peptides and Proteins, P. Lloyd-Williams, F. Albericio, E. Giralt, CRC Press, Boca Raton, 1997; Bioorganic Chemistry: Peptides and Proteins, S. M. Hecht, Ed., Oxford Press, Oxford, 1998, are used. One skilled in the art may develop equivalent means or reactants without the exercise of inventive capacity and without departing from the scope of the invention.

It will be understood that many variations can be made in the compositions and procedures herein described while still remaining within the bounds of the present invention. Likewise, it is understood that, due to known structural or chemical similarities such as polarity, bulk, or orientation between amino acid side chains, peptide sequences with amino acids or replacement structures equivalent to those disclosed herein will retain similar function. Thus, for example, a tetrapeptide repeat such as NPNA may be replaced with a closely related NPNV repeat, and a DPNV repeat may be replaced by a DPNA repeat while still serving the purposes of the invention. Similarly, the coupling of groups in aminoproline and glutamate is to be understood as only one illustrative example of the invention which could be replaced by many equivalent crosslinks. It is the intention of the inventors that such variations are included within the scope of the invention.

### EXAMPLE 1

This example demonstrates the synthesis of the large-loop conformationally constrained, template-bound peptidomimetic cyclo-(Asn-Ala-Asn-Pro-Asn-Ala-Asn-Pro-Asn-Ala-Template). The synthesis of a linear precursor was performed on Tentagel S-AC resin. The first amino acid, Fmoc-Ala-OH (143 mg, 3. eq.) was attached to the resin (0.5 g, 0.29 mmol/g) in dichloromethane and pyridine (1:1, 2 ml) using 2-chloro-1,3-dimethylimidazolidinium hexafluorophosphate (CIP) (6 eq.). The resin was then washed successively with dichloromethane, methanol and dichloromethane. The Fmoc-group was then removed with piperidine in dimethylformamide (DMF) (20%, v/v). The standard Fmoc-chemistry was then used to assemble the following peptide chain with side-chain protecting groups: H-Asn(Mtt)-Pro-Asn(Mtt)-Ala-Template(OtBu)-Asn(Mtt)-Ala-Asn(Mtt)-Pro-Asn(Mtt)-Ala-O-resin. (Mtt=4-methyltrityl). This peptide was cleaved from the resin with 1% v/v TFA in dichloromethane. The cleavage solution was neutralized with pyridine, washed with water and evaporated to dryness. The resulting product was dissolved in dimethylformamide (DMF) (1 mg/ml) and treated with O-(7-azabenzotriazol-1-yl)-N,N,N′,N′-tetramethyluronium hexafluorophosphate (HATU, 3 eq.), 1-hydroxy-7-azabenzotriazole (HOAt, 3 eq.) and diisopropylethylamine(DIEA, 1%, v/v) overnight at room temperature. The solvent was then evaporated, the residue redissolved in dichloromethane, extracted with acetonitrile/water, and then evaporated to dryness. The product was deprotected by dissolving in trifluoroacetic acid (TFA)-triisopropylsilane (TIS)-water (95:2.5:2.5) for 2 h at room tempertaure. The sample was then evaporated to dryness and the product purified by HPLC on a C18 reverse phase column using a gradient of 5-50% acetonitrile in water+0.1% TFA. ES-mass spectrum showed m/z 1267.7 (100%, M+Na).

### EXAMPLE 2

This example shows the modification of the conformationally constrained largeloop peptidomimetic to cyclo-(Asn-Ala-Asn-Pro-Asn-Ala-Asn-Pro-Asn-Ala-Template)-β-alanine conjugate by addition of an alanine linker for coupling to PE or PE′. The peptidomimetic (see above) (20 mg) and β-alanine t-butyl ester (18 mg, 6 eq.) in DMF (1 ml), with HATU (18 mg, 3 eq.), HOAt (6.6 mg, 3 eq.) and DIEA (33 μl), 12 eq.) were stirred at room temperature for 4 h. The solution was then evaporated to dryness, and the product was purified by HPLC (C18 reverse phase column using a gradient of 5-50% acetonitrile in water+0.1% TFA). ES-mass spectrum showed m/z 1394.9 (M+Na). This product was treated with TFA:water (4 ml, 3:1) for 1.5 h. The solution was then evaporated and the product purified by HPLC (C18 reverse phase column using a gradient of 5-50% acetonitrile in water+0.1% TFA). ES-mass spectrum showed m/z 1338.0 (M+Na).

### EXAMPLE 3

This example demonstrates the synthesis of cyclo-(Asn-Ala-Asn-Pro-Asn-Ala-Asn-Pro-Asn-Ala-Template)-B-alanine-PE′ by conjugation of the peptidomimetic to PE. To the product of the previous Example, (10 mg), HATU (6 mg, 2 eq.), HOAt (2 mg, 2 eq.), and DIEA (8 μl) in N-methylpyrrolidone (NMP, 0.7 ml) was added rac-1-palmitoyl-3-oleoyl-phosphatidylethanolamine (PE′) (11 mg, 2 eq.) in dichloromethane (0.5 ml). The solution was stirred overnight at room temperature. The solvent was then evaporated and the product was purified by chromatography on silica gel eluting with chloroform:methanol:acetic acid:water (9:6:0.5:05) to give the cyclo-(Asn-Ala-Asn-Pro-Asn-Ala-Asn-Pro-Asn-Ala-Template)-β-alanine-PE′ (11 mg). ES-mass spectrum showed m/z 2039.5 (M+Na).

### EXAMPLE 4

This example demonstrates how antibody responses elicited by a conformationally constrained peptidomimetic formulated with alum or with IRIVs are compared: First, antibody responses elicited by the MAP construct with the conformationally constrained, template-bound small loop peptidomimetic adsorbed onto alum, and by IRIVs loaded with the peptidomimetic linked to PE, were compared in BALB/c mice. After three immunizations, sera from all immunized animals contained mimetic-specific antibodies, as demonstrated by enzymelinked immunosorbent assays (ELISAs) with immobilized peptidomimetic-MAP (FIG. 9). None of the sera exhibited cross-reactivity with the template-MAP construct 8 (see FIG. 10), which indicates that the immunogenicity of the template itself was negligible.

### EXAMPLE 5

This example shows the cross-reactivity of anti-peptidomimetic antibodies with the parasite protein: The binding of antibodies to the CS protein was analyzed by immunofluorescence assays (IFAs) with P. falciparum sporozoite preparations (FIG. 9). The IRIV formulation elicited significant anti-sporozoite responses in all animals immunized. In contrast, half of the animals immunized with the alum formulation generated no detectable antisporozoite antibody response and in the others the IFA titers were very low. The IRIV formulation thus elicited a higher proportion of parasite-binding antibodies among the total antimimetic antibodies than the alum formulation (compare FIGS. 11A and B).

### EXAMPLE 6

This example demonstrates the immunogenicity of the large loop template bound cyclic peptidomimetic coupled to IRIV. In a second series of experiments (Moreno, R. et al., ChemBioChem 2001, 2, 838-843) the immunogenicity of the larger loop conformationally constrained, template-bound peptidomimetic was analyzed. After three immunizations with IRIV-loaded peptidomimetic, significant ELISA and IFA titers were seen in sera from all immunized mice (FIG. 9). While the ELISA titers were only slightly higher than those elicited by IRIV-loaded small loop conformationally constrained, template-bound peptidomimetic, the IFA titers were significantly higher (compare FIGS. 11A and B). The large loop conformationally constrained, template-bound mimetic was thus superior to the smaller loop peptidomimetic in eliciting a high proportion of parasite cross-reactive antibodies. None of the antisera against the large loop conformationally constrained, template-bound peptidomimetic cross-reacted with the template-MAP conjugate in an ELISA.

### EXAMPLE 7

This example demonstrates the binding properties of peptidomimetic-specific monoclonal antibodies: ELISA titers of sera of individual peptidomimetic-IRIV immunized mice did not correlate strictly with IFA titers, (FIG. 9; FIG. 12). This suggested that only a subset of antibodies elicited against the peptidomimetics cross-reacted with the CS protein on the cell surface of the sporozoites. To investigate this further, monoclonal antibodies (mAbs) against both the smaller loop and larger loop conformationally constrained, template-bound peptidomimetics were generated. Three hybridoma cell lines secreting mAb against the smaller loop template-bound peptidomimetic and six lines secreting mAb against the larger loop template-bound peptidomimetic were isolated. The cross-reactivities of the mAb produced by the three anti-small-loop template-bound peptidomimetic-specific clones (designated mAbs 1.7, 1.15, and 1.26) and by the six anti-larger loop template-bound peptidomimetic specific clones (designated mAbs 2.1, 3.1, 3.2, 3.3, 3.4, and 3.5) were analyzed (FIG. 13). One of the mAbs (mAb 1.26) against the small loop template-bound peptidomimetic and four of the mAbs (2.1, 3.1, 3.2, and 3.3) against the larger loop template-bound peptidomimetic cross-reacted with P. falciparum sporozoites in IFAs (FIG. 14). All mAbs bound to the peptidomimetic used for immunization but not to the respective second mimetic or to the template structure (FIG. 13).

### EXAMPLE 8

This example shows how molecular modeling is used to design longer, more complex conformationally constrained template-independent peptidomimetics: Using the backbone φ/ψ angles for Asn³-Asn⁷ taken from earlier models of the template-bound larger loop mimetic (Pfeiffer, B. et al., Chimia 55 (2001), 334-339), a linear peptide was built with the sequence Ac-(NPNA)₅-NH₂ wherein the helical turn conformation with the appropriate backbone φ/ψ angles was also tandemly repeated. The resulting model of this conformationally constrained, template-independent peptidomimetic (FIG. 5) was stable in molecular dynamics (MD) simulations in water solvent, and adopted the expected repetitious supersecondary structure shown in FIG. 6A. It is this supersecondary structure which may be close to the preferred conformation of the NPNA-repeat region in the native CS protein.

To confirm this prediction, an approach was designed to stabilize this supersecondary structure by appropriate cross-linking of the peptide backbone (FIG. 4), in order to investigate the ability of the resulting cross-linked peptidomimetic to elicit antibodies that recognize the native CS protein on sporozoites. Examination of molecular models suggested that a suitable cross-link could be formed by introducing an amino group at the β-position of Pro⁶ and amide coupling to the spatially adjacent side chain carboxyl of Glu as a replacement for Ala¹⁶, i.e. as indicated in FIG. 4. A model of this cross linked peptidomimetic was constructed, and the model also remained in the expected conformation during molecular dynamics (MD) simulations in water (FIG. 6B).

### EXAMPLE 9

This example shows the design and synthesis of the template used to construct conformationally constrained template-bound peptidomimetics: the template (Bisang, C. et al., J.Am.Chem.Soc.1998, 120, 7439-7449 and WO 01/16161) can be readily synthesized on a gram scale. The alkylation of Me₃Si-protected N-Z-4-hydroxyproline methyl ester can be performed conveniently on a large scale and after removal of the temporary Si-protecting group gives the desired diastereomer in a bout 2:1 (3:3′) excess. After a Mitsunobu reaction (Mitsunobu, O.; Wada, M.; Sano, T. J. Am. Chem. Soc. 1972, 94, 679-680) and removal of the Z protecting group, the desired steroisomer could be obtained by recrystallization, without the need for large scale chromatography. The relative configuration of the stereoisomer was confirmed by steady-state NOE-difference experiments. The coupling of the stereoisomer with Fmoc-Asp(allyl)-OH proceeded in good yield using DCC and HOAt for activation. The resulting dipeptide could then be cyclized directly, and exchange of protecting groups yielded the template in a form suitable for solid-phase peptide synthesis. To assemble the cyclic antigen on a solid-phase, the template was coupled to Tentagel S-AC resin through the free carboxylic acid, and the linear peptide chain was then assembled using standard Fmoc-chemistry (Atherton, E.; Sheppard, R. C. Solid-phase peptide synthesis—a practical approach; IRL Press; Oxford, 1989). The allyl ester of the template was cleaved using Pd⁰, and cyclization was accomplished with BOP and DIEA in NMP-DMSO. The products of the cyclization were analyzed by reverse-phase HPLC following cleavage from the resin and deprotection with TFA.

### EXAMPLE 10

This example shows the conformation of the large-loop template bound cyclic peptidomimetic: To determine which conformations were preferred in the template-bound cyclic peptide antigen, the conformation of the conformationally constrained, template-bound larger loop peptidomimetic in aqueous solution was investigated by NMR spectroscopy at 290 and 300 K and pH 5.0. The chemical shift assignments were made by standard methods (Wuethrich, K. NMR ofproteins and nucleic acids; Wiley-Interscience: New York, 1986). The one-dimensional NMR spectra indicated the presence of one major conformer and two minor ones (in a ratio of 77:15:8) with the latter two most probably arising due to cis-trans isomerism at Asn-Pro peptide bonds, in analogy to earlier studies (H. J. Dyson, A. C. Satterhwait, R. A. Lemer, P. E. Wright, Biochemistry 1990, 29, 7828; C. Bisang. L. Jiang, E. Freund, F. Emery, C. Bauch, H. Matile, G. Pluschke, J. A. Robinson, J. Am. Chem. Soc. 1998, 120, 7439). The minor forms were not considered further. Although the peptide backbone groups (NH, C(α)H) could be assigned, extensive overlap prevented residue-specific assignments of side-chain Asn resonances. This together with a sparcity of long-range NOEs thwarted attempts to calculate solution structures based on NOE restraints.

Nevertheless, NOESY spectra revealed strong d_{NN}(i,i+1) connectivities between Asn⁵ and Ala⁶, as well as between Ala⁶ and Asn⁷. These, together with high field shifted resonances and a relatively low temperature coefficient for the Ala⁶ NH group (δ=7.82 and δ/Δ=3.7 ppb K⁻¹) suggest a β turn is formed by the four residues Asn³-Pro⁴-Asn⁵-Ala⁶. A β-turn structure, however, may not be the whole story. The d_{NN}(i,i+1) connectivities show that the Ala⁶ NH group is close to the peptide NH group of the preceding Asn (as expected in a βturn) and the following Asn residue. This could occur if the Ala⁶ residue is in the α region of φ/ψ space, with the Asn³ CO moiety within (or close to) hydrogen-bonding distance of both the Ala⁶ NH and the Asn⁷ NH groups as shown in a model in FIG. 3. This leads to the intriguing possibility that conformations are present in which a perhaps distorted β turn is extended by one residue to create a five-residue conformational unit (NPNAN) with Ala in a helical state.

### EXAMPLE 11

This example demonstrates the ynthesis of a larger, complex conformationally constrained, template-independent peptidomimetic: The required orthogonally protected (2S,3R)-3-aminoprolinewas prepared from the known β-lactam as shown in FIG. 7. The chemistry is straightforward, and the synthesis proceeds in good yields. The required cross-linked peptidomimetic was prepared by solid phase synthesis methods, as outlined in FIG. 8. The 20-mer peptide 10 was assembled using Fmoc-chemistry. Cleavage from the resin and removal of side-chain protecting groups proceeded in one step to afford 11. The key backbone coupling of the Apro⁶ and Glu¹⁶ side chains was then achieved in a remarkably clean and high yielding cyclization in DMF with HATU. Monitoring the reaction by HPLC showed essentially quantitative cyclization of the precursor (data not shown). This high efficiency probably reflects the fact that the required conformation is strongly preferred by the peptide backbone. Finally, the 20-mer was acetylated for conformational studies by NMR, and was also coupled via a succinate linker to a regioisomer of phosphatidyl ethanolamine (PE′, 1-palmitoyl-3-oleoyl-phosphatidylethanolamine) to afford the conjugate ready for incorporation into an IRIV.

### EXAMPLE 12

This example demonstrates the synthesis of the linear peptide in the construction of a larger, more complex conformationally constrained template-independent peptidomimetic: The resin (0.5 g Rink Amide MBHA (Novabiochem) was swelled for 1 h in DMF (6 ml/g). The DMF was then removed (decantation). Then Fmoc group was removed using 20% piperidine/DMF for 45 min. Then 2 eq of Fmoc-Ala-OH, 3 eq 1-hydroxybenzotriazole (HOBt), and 3 eq. O-(benzotriazol-1-yl)-N,N,N′,N′-tetramethyluronium hexafluorophosphate (HBTU) were added together with 40 μl DIEA in 4 ml DMF. The mixture was stirred overnight at room temperature. For capping of any remaining residual free amino sites benzoic anhydride (679 mg, 3.0 mmol) in 3 ml DMF and 1% DIEA was added and shaken for 1 h. The linear peptide was then assembled using an Applied Biosystems ABI-433A Peptide Synthesizer attached to a Perkin Elmer 785A UV/Vis detector. The peptide was synthesized on a 0.25 mmol scale, using 4 eq of Fmoc-amino acid activated with HBTU/HOBt. The amino acids used were: Fmoc-Asn(Mtt)-OH, Fmoc-Pro-OH, Fmoc-Ala-OH, Fmoc-Glu(tBu)-OH.

### EXAMPLE 13

This example shows how the incorporation of a proline residue specially adapted for internal crosslinking of the conformationally constrained, template-independent peptidomimetic is used to cyclize the peptidomimetic. For the coupling of Fmoc-(2S,3R)-3-amino(Boc)proline-OH the resin was taken out of the synthesizer and 4 eq. of the amino acid was added together with 4 eq. HOAt, 4 eq. HATU, 8 eq. DIEA in 4 ml DMF. This mixture was swirled for 2 h. The Kaiser test showed completion of the coupling and the rest of the linear peptide was assembled using again the peptide synthesizer. In the final step the resin was washed with NMP, then dichloromethane, leaving the N-terminal Fmoc protection group intact. The cleavage of the linear peptide from the resin was carried out using TFA containing 2.5% TIS and 2.5% water. After removal of the TFA the peptide was precipitated using diisopropyl ether and dried overnight, yielding 450 mg (64%) of the linear peptide. ESI-MS (MeOH/H₂O+0.5% AcOH): 1159 ([M+NaH]²⁺); 1148 ([M+2H]²⁺). For cyclization the crude linear peptide was stirred overnight together with 4 eq HOAt, 4 eq HATU, in DMF with 1% DIEA. The solution was then concentrated and the product was purified by HPLC (Vydac 218TP1010™ C₁₈ column, gradient 5-100% acetonitrile in water+0.1% TFA over 20 min). Yield: 182.7 mg (41%). ESI-MS (MeOH/H₂O+0.5% AcOH): 2299 ([M+Na]⁺); 2278 ([M+H]⁺). The Fmoc group was removed by stirring the peptide in 20% piperidine/DMF for 10 min. The solution was concentrated and peptide was precipitated using diisopropyl ether and dried overnight, yielding 142.7 mg (99%) of the internally crosslinked peptidomimetic. ESI-MS (MeOH/H₂O+0.5% HCOOH): 2077 ([M+Na]⁺); 2055 ([M+H]⁺).

### EXAMPLE 14

This example demonstrates how the larger, more complex conformationally constrained, template-independent peptidomimetic is linked to PE. The linear peptide (40 mg) was dissolved with HOAt (16 mg) and HATU (28 mg) in NMP (4 ml) and DMF (2 ml). Then a solution of PE′-CO—(CH₂)₂—COOH (24 mg) in dichloromethane (1 ml) was added together with DIEA (70 μl, 1%) and the mixture stirred for 20 h. The solution was concentrated and the resulting residue was purified over a C₄-catridge (Vydac Bio-Select™ 214TB213) using first 20% then 50% acetonitrile in water as the eluent. The 50% fraction was then lyophilised. Yield: 23.3 mg (42%). ESI-MS (MeOH/H₂O+1% HCOOH): 1439 ([M+NaH]²⁺); 1428 ([M+2H]²⁺).

### EXAMPLE 15

This example shows how a PE stereoisomer can be linked to succinic acid to yield PE′-CO—(CH₂)₂—COOH (PE′-succinic acid conjugate). The PE′ (50 mg) was dissolved in dichloromethane (2 ml) and treated with succinic anhydride (1.1 eq.) and N,N-4-dimethylaminopyridine (DMAP) (1.1 eq.) at room temperature overnight. The solution was then washed with water and evaporated to dryness. The product was used without further purification.

### EXAMPLE 16

This example shows the conformational studies of the conformationally constrained, template-independent peptidomimetic: To determine which conformations were preferred in the conformationally constrained peptide antigen, its conformation in aqueous solution was investigated. The preferred solution conformation of the peptidomimetic was studied by NMR and MD methods in aqueous solution at pH 5 and 293K. The 1D ¹H NMR spectra indicated the presence of a major conformer and two minor ones (ratio 80:14:6), the latter two most likely arising due to cis-trans isomerism at Asn-Pro peptide bonds. The minor forms were not considered further. A full assignment of the ¹H spectrum of the major form was complicated by chemical shift overlap, particularly of the Asn H—C(β) resonances. However, the peptide backbone HN, H—C(α) resonances could be assigned unambiguously.

2D NOESY spectra showed strong d_{NN}(i,i+1) connectivities between the peptide NH groups of Asn⁷ and Ala⁸ as well as Ala⁸ and Asn⁹ in the first helical turn, Asn¹¹ and Ala¹² as well as Ala¹² and Asn¹³ in the next helical turn, and between Asn¹⁵ and Glu¹⁶ as well as Glu¹⁶ and Asn¹⁷ in the last helical turn. These together with the observation of long range NOEs between Pro H—C(a) (i+1) and Ala HN (i+3), provide evidence for three relatively stable helical turns formed by the residues Asn⁵-Asn⁹, Asn⁹-Asn¹³, and Asn¹³-Asn¹⁷.

Average solution structures for the peptidomimetic were calculated using NOE-derived distance restraints by dynamic simulated annealing (SA) and molecular dynamics (MD) simulations, using methods described earlier (Bisang, C.; Weber, C.; Robinson, J. A. Helv. Chim. Acta 1996, 79, 1825-1842). The resulting average structures revealed a common core comprising the anticipated three helical turns from Asn⁵-Asn ⁷, with higher flexibility in the regions of the N-and C-termini (FIG. 6C). The backbone conformation of the central region, however, corresponded closely to the expected supersecondary structure deduced for models of the peptidomimetic (FIG. 3A and 3B). It was concluded, therefore, that although the mimetic is not rigid, it can adopt a supersecondary structure comprising three interlinked helical turns, each based on the (NPNAN) motif.

### EXAMPLE 17

This example shows the preparation of mimetic-loaded virosomes. For the preparation of PE-mimetic-IRIV, a solution of 4 mg purified Influenza A/Singapore hemagglutinin was centrifuged for 30 min at 100 000 g and the pellet was dissolved in 1.33 ml of PBS containing 100 mM OEG (PBS-OEG). 32 mg phosphatidylcholine (Lipoid, Ludwigshafen, Germany), 6 mg phosphatidylethanolamine and the PE-mimetics were dissolved in a total volume of 2.66 ml PBS-OEG. The phospholipids and the hemagglutinin solution were mixed and sonicated for 1 min. This solution was then centrifuged for 1 hour at 100,000 g and the supernatant was sterile filtered (0.22 μm). Virosomes were then formed by detergent removal using BioRad SM BioBeads (BioRad, Glaffbrugg, Switzerland).

### EXAMPLE 18

This example shows the immunogenicity studies for the peptidomimetic-IRIV constructs: BALB/c mice were preimmunized intramuscularly with commercial whole virus influenza vaccine (0.1 ml; Inflexal Berna, Berna Products, Bern, Switzerland) on day 21. Starting on day 0, they received at three-weekly intervals three doses of either the small loop, conformationally constrained template-bound peptidomimetic-MAP construct adsorbed to alum (allhydrogel 85), the small loop, conformationally constrained, template-bound peptidomimetic linked to IRIV, or the larger loop, conformationally constrained, template-bound peptidomimetic linked to IRIV intramuscularly at doses of 50 μg of mimetic. Blood collected two weeks after the third immunization was analyzed by ELISa and IFA.

### EXAMPLE 19

This example shows how immunological studies with the template-bound larger loop peptidomimetic are performed: In a series of experiments the immunogenicity of the larger loop, conformationally constrained, template-bound cyclic peptidomimetic was analyzed. After three immunizations with IRIV-loaded peptidomimetic, significant ELISA and IFA titers were seen in sera from all immunized mice (FIG. 9). While the ELISA titers were only slightly higher than those elicited by IRIV-loaded small loop template-bound peptidomimetic, the IFA titers were significantly higher (compare FIGS. 11A and B). The larger loop conformationally constrained, template-bound peptidomimetic was thus superior to the small loop peptidomimetic in eliciting a high proportion of parasite cross-reactive antibodies. None of the anti-large loop template-bound-IRIV antisera cross-reacted with the template-MAP conjugate in an ELISA (data not shown).

### EXAMPLE 20

This example demonstrates the generation of antipeptidomimetic monoclonal antibodies and the examination of the binding properties of peptidomimetic-specific mAb. ELISA titers of sera of individual mimetic-IRIV immunized mice did not correlate strictly with IFA titers (FIG. 9). This suggested that only a subset of antibodies elicited against the peptidomimetics cross-reacted with CS-protein on the cell surface of the sporozoites. To investigate this firther, we generated monoclonal antibodies (mAbs) against both the small and larger loop template-bound peptidomimetics. Three hybridoma cell lines secreting monoclonal antibodies specific for the small loop conformationally constrained, template-bound peptidomimetic, and six lines secreting monoclonal antibodies specific for the larger loop, conformationally constrained template-bound peptidomimetic were isolated. The crossreactivities of the monoclonal antibodies produced by the three clones specific for the small loop template-bound peptidomimetic (designated 1.7, 1.15 and 1.26) and by the six clones specific for the larger loop, template-bound peptidomimetic (designated 2.1, 3.1, 3.2, 3.3, 3.4 and 3.5) were analyzed (FIG. 13). One of the monoclonal antibodies specific for the small loop template-bound peptidomimetic (mAb 1.26) and four of the monoclonal antibodies specific for the larger loop template-bound peptidomimetic (2.1, 3.1, 3.2 and 3.3) cross-reacted with P. falciparum sporozoites in IFA (FIG. 5). All monoclonal antibodies bound to the peptide mimetic used for immunization, but not to the respective second mimetic or to the template structure (FIG. 13).

### EXAMPLE 21

This example shows how Enzyme-Linked Immunosorbent Assays (ELISA) were performed. ELISA microtiter plates (Immunolon 4B, Dynatech, Embrach, Switzerland) were coated at 4° C. overnight with 50 ml of a 5 mg/ml solution of peptidomimetic-MAP constructs in PBS, pH 7.2. Wells were then blocked with 5% milk powder in PBS for 1 h at 37° C. followed by three washings with PBS containing 0.05% Tween-20. Plates were then incubated with twofold serial dilutions of mouse serum or hybridoma cell supernatants in PBS containing 0.05% Tween-20 and 0.5% milk powder for 2 h at 37° C. After washing, the plates were incubated with alkaline phosphatase-conjugated goat anti mouse IgG (g-chain specific) antibodies (Sigma, St. Louis, Mo.) for 1 h at 37° C. and then washed. Phosphatase substrate (1 mg/ml p-nitrophenyl phosphate (Sigma)) in buffer (0.14% Na2CO3, 0.3% NaHCO3, 0.02% MgCl2, pH 9.6) was added and incubated at room temperature. The optical density (OD) of the reaction product was recorded after appropriate time at 405 nm using a microplate reader (Titertek Multiscan MCC/340, Labsystems, Finland). Titration curves were registered and analyzed using GENESIS LITE 2.16 software (Life Sciences Ltd., Basingstoke, UK). Effective dose 20% values (ED20%) were calculated for each curve and the corresponding titers were set as endpoint titers.

### EXAMPLE 22

This example shows how immunofluorescence assays were performed to assess cross-reactivity of the antibodies obtained. Air-dried unfixed P. falciparum salivary gland sporozoites (strain NF54) attached to microscope glass slides were incubated in a moist chamber for 20 min at 37° C. with serum diluted in PBS. The slides were then washed five times with PBS containing 0.1% bovine serum albumin (PBS-BSA) and dried. FITC-labeled goat anti-mouse IgG (Fab-specific) antibodies (Sigma), optimally diluted in PBS containing 0.1 g/L Evans-Blue (Merck, Germany), were added. After incubation for 20 min at 37° C. the slides were again washed five times with PBS-BSA, dried, mounted with glycerol, and covered with a cover slide. A Leitz Dialux 20 microscope using 12.5/18 ocular and a 40×/1.30 oil fluorescence 160/0.17 objective was used to detect fluorescence staining at 495 nm excitation and 525 nm emission wavelengths.

What is claimed is:

1. A composition comprising a conformationally constrained peptidomimetic comprising at least one tetrapeptide repeat selected from the group consisting of NPNA, DPNA, NPNV, and DPNV;
wherein, if the tetrapeptide repeat is NPNA, the peptidomimetic comprises at least two NPNA repeats.

2. The composition of claim 1 wherein a template conformationally constrains the peptidomimetic.

3. The composition of claim 1 wherein a crosslink conformationally constrains the peptidomimetic.

4. A composition comprising a conformationally constrained peptidomimetic of the sequence NPNAN-X-NANPNANPN-Y-NPNA wherein X is selected from the group consisting of Aminoproline and Cysteine, and wherein Y is selected from the group consisting of Glutamate, Aspartate, and Cysteine.

5. The composition of claim 4, wherein a crosslink between Aminoproline⁶ and Glutamate¹⁶ conformationally constrains the peptidomimetic.

6. A template-bound conformationally constrained peptidomimetic comprising the sequence NANPNANPNA having a helical turn conformation.

7. A method of synthesizing conformationally constrained template-bound peptidomimetics comprising the steps of:
(a) assembling a linear peptide comprising a plurality of tetrapeptide repeats selected from the group consisting of NPNA, DPNA, NPNV, and DPNV; and
(b) cyclizing the linear peptide to a template, wherein if the tetrapeptide repeat is NPNA, the peptidomimetic comprises at least two NPNA repeats.

8. A method of synthesizing internally cross-linked peptidomimetics comprising the steps of:
(a) obtaining a polypeptide comprising a plurality of tetrapeptide repeats selected from the group consisting of NPNA, DPNA, NPNV, and DPNV;
(b) determining a suitable spatial position for crosslinking by molecular modeling;
(c) providing amino acids with appropriate modifications for crosslinking;
(d) synthesizing a linear peptide comprising the amino acids with appropriate modifications for crosslinking; and
(e) assembling the linear peptide into crosslinked form.

9. The conformationally constrained peptidomimetic produced by the method of claim 6.

10. The conformationally constrained peptidomimetic produced by the method of claim 7.

11. A method of making conformationally constrained peptidomimetics comprising the steps of:
(a) identifying an amino acid sequence having a plurality of peptide repeats;
(b) molecular modeling the conformation of the amino acid sequence;
(c) determining a suitable position for a stabilizing crosslink;
(d) assembling a conformationally constrained peptidomimetic.

12. An immunopotentiating reconstituted influenza virosome comprising a plurality of at least one kind of conformationally constrained peptidomimetic comprising at least one tetrapeptide repeat selected from the group consisting of NPNA, DPNA, NPNV, and DPNV.

13. A composition comprising a plurality of the immunopotentiating reconstituted influenza virosome of claim 12.

14. A method of generating sporozoite-specific antibodies comprising the step of:
(a) administering to a subject immunopotentiating reconstituted influenza virosomes comprising a plurality of at least one kind of conformationally constrained peptidomimetic comprising at least one tetrapeptide repeat selected from the group consisting of NPNA, DPNA, NPNV, and DPNV;
(b) isolating the antibodies generated; and
(c) assaying the antibodies generated for cross-reactivity with malaria sporozoites.

15. A method of generating sporozoite-specific antibodies comprising the steps of:
(a) administering to a subject a composition comprising a conformationally constrained peptidomimetic comprising at least one tetrapeptide repeat selected from the group consisting of NPNA, DPNA, NPNV, and DPNV;
(b) isolating the antibodies generated; and
(c) assaying the antibodies generated for cross-reactivity with malaria sporozoites.

16. An antibody generated by the method of claim 14.

17. An antibody generated by the method of claim 15.
